(19) United States
(12) Patent Application Publication
(10) Pub. No.: US 2011/0262895 A1
(43) Pub. Date: Oct. 27, 2011

(54) METHODS FOR THE DIAGNOSIS OF VARICELLA ZOSTER VIRUS INFECTION

(76) Inventors: Robert E. Harding, Missouri City, TX (US); Randall J. Cohrs, Littleton, CO (US); Donald H. Gilden, Greenwood Village, CO (US); Duane L. Pierson, Houston, TX (US); Satish K. Mehta, Friendswood, TX (US)

(21) Appl. No.: 13/057,399

(22) PCT Filed: Aug. 6, 2009
(86) PCT No.: PCT/US09/52992

§ 371 Date: Jul. 15, 2011

(30) Foreign Application Priority Data
Aug. 7, 2008 (US) 61087045

(51) Int. Cl. C12Q 1/70; C12M 1/34

(52) U.S. Cl. 435/5; 435/287.3

(57) ABSTRACT

The present invention relates to methods and devices for the rapid assessment of saliva for the presence of varicella zoster virus (VZV) particles. The methods and devices permit rapid, simple and cost-effective diagnosis of primary VZV infection.

This application claims benefit of priority to U.S. Provisional Application Ser. No. 61/087,045, filed Aug. 7, 2008, the entire contents of which are hereby incorporated by reference.

### BACKGROUND OF THE INVENTION

I. Field of the Invention

The invention relates to the fields of immunology, medicine and infectious disease. In particular, the invention relates to methods and devices for the rapid detection of varicella zoster virus in saliva.

II. Related Art

Varicella zoster virus (VZV) is one of eight herpes viruses known to infect humans (and other vertebrates). Primary VZV infection, usually transmitted through airborne respiratory droplets and smear infection of the virus filled lesions of infected people to susceptible contacts, results in chickenpox (varicella), which may rarely result in complications including encephalitis or pneumonia.

The seroprevalence of antibodies to VZV increases from 30% in children by the age of 1 year to about 95% towards the age of 10 years and remains then lifelong at the same level. In the U.S. the cumulative incidence is 20%, or an annual rate of 3-5 cases/1000 persons. Internationally no accurate data is available, but the incidence seems to be similar to that in the U.S. The incidence increases with age and impaired immune status.

Even when clinical symptoms of chickenpox have resolved, VZV remains dormant in the nervous system of the infected person (virus latency), in the trigeminal and dorsal root ganglia. In about 10-20% of cases, VZV reactivates later in life producing a disease known as herpes zoster or shingles. Serious complications of shingles include postherpetic neuralgia, zoster multiplex, myelitis, herpes ophthalmicus, or zoster sine herpete.

The clinical presentations of varizella or zoster are so characteristic that laboratory confirmation is rarely required. However, specific laboratory diagnosis is required for atypical presentations, as found in immunocompromised patients, infections during pregnancy, diseases of the CNS, pneumonia, and in distinguishing between HSV infections and Herpes Zoster.

Direct detection methods from the lesions, such as cytology and electron microscopy (EM), cannot distinguish between VZV and HSV. Immunofluorescence cytology is more sensitive than EM but is more labor intensive and requires greater technical expertise. PCR assays for VZV are available and have been reported to be of use in the diagnosis of VZV meningoenzephalitis from CSF specimens. The isolation of VZV from fibroblast culture is considered the definitive method for diagnosing VZV infections. However, virus isolation is rarely carried out because of the long length of time required for a result to be available (up to 15 days) and the low sensitivity of 40% to 50%. Thus, sensitive, rapid, simply and cost-effective detection tools for VZV infections are therefore greatly needed.

### SUMMARY OF THE INVENTION

Thus, in accordance with the present invention, there is provided a method of detecting an active varicella zoster virus (VZV) infection in a human subject comprising (a) obtaining a saliva sample; and (b) detecting VZV virus particles in the sample. Detecting comprises an immunoassay or a nucleic acid-based assay, such as PCR. An immunoassay can use a first antibody to a first VZV antigen, and a second antibody to a second VZV antigen. The first antibody may be linked to a bead and the second antibody may be linked to a detectable marker. The bead may be sequestered in a reaction chamber, and the detectable marker may be detected in the chamber when VZV is present. The bead may be a magnetic or paramagnetic bead. The detectable marker may be an enzyme, a fluorescent label, or a chemilluminescent label. The reaction chamber may be a syringe, and obtaining may comprise positioning the syringe in a subject's mouth and filling the syringe with the saliva sample. The subject may suffer from primary or recurrent VZV infection. The method may further comprise transferring the saliva sample into a detection chamber, such as a column. The detection chamber may be attached to a disposal reservoir. The method may also further comprise introducing a wash solution into the detection chamber. The method may further comprise generating a detectable positive reaction in the reaction chamber.

In another embodiment, there is provided a kit for the detection of virus in a fluid comprising (a) a reagent chamber comprising a first antibody conjugated to a microbead, and a second antibody conjugated to a detectable marker; (b) a detection chamber comprising a capture agent for binding the microbead; and (c) a disposal reservoir; wherein the reagent chamber and the detection chamber support fluid transfer therebetween, and the detection chamber and the disposal reservoir support fluid transfer therebetween. The first reagent chamber may be contained within a syringe. The detection chamber may comprise a column. The bead may be a magnetic bead, and the capture agent may be a metal support. The kit may further comprise a wash solution and/or a detection solution, either or both being packaged in a syringe. The kit may further comprise a mouth piece that supports operable connection with the reaction chamber. The kit may further comprise a magnetic separator. The capture agent may bind to a virus, such as a herpesvirus, such as varicella zoster virus. The capture agent may bind to a bacterium, a fungus or a parasite, an environmental or industrial waste product or toxin, or a bioharzard or bioterrorism agent.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIG. 1—Components of Rapid Detection Device. Reagent chamber/syringe 1; reaction chamber 2; waste disposal chamber/bag 3; wash syringe 4; detection syringe 5; mouthpiece 6; a magnetic separator 7.

FIGS. 2A-G—Steps for the use of the Rapid Detection Device. (FIG. 2A) The reagent syringe is used to capture the sample from the subject. (FIG. 2B) The sample is introduced into a reaction chamber and waste is driven into a disposal bag. (FIG. 2C) Wash flued is introduced into the reaction chamber and waste is driven into the disposal bag. (FIG. 2D) Unbound material is washed from the reaction chamber into the disposal bag. Antibiodies against a viral conjugated to microbeads retain the virus on the column matrix, and secondary anti-viral antibodies conjugated to a marker (HRP) bind to the retained virus. (FIG. 2E) Luminol in a detection syringe is introduced into the reaction chamber and waste is driven into the disposal bag. (FIG. 2F) Molecular reaction in a positive sample. (FIG. 2G) Graphic view of positive and negative reaction in the device.

### DETAILED DESCRIPTION OF THE INVENTION

Varizella-Zoster Virus (VZV) or human herpesvirus 3 (HHV3) is a highly contagious virus that affects people worldwide. VZV primary infections normally occur during childhood with moderate symptoms in immunocompetent patients, but immunosuppressed patients may have serious complications, such as CNS pathology, pneumonia, and secondary bacterial infections. While varizella or zoster may be identified by clinical/symptomatic characteristics, specific laboratory diagnosis may be required for atypical presentations, as found in immunocompromised patients, infections during pregnancy, diseases of the CNS, pneumonia, and in distinguishing between HSV infections and Herpes Zoster. In addition, early identification (prior to symptoms) of affected subjects in an outbreak is of great value.

However, as explained above, there are considerable limitations in diagnostic methods for VZV infection. Direct detection methods from the lesions, such as cytology and electron microscopy (EM), cannot distinguish between VZV and HSV. Immunofluorescence cytology is more sensitive than EM, but is more labor intensive and requires greater technical expertise. PCR assays for VZV are available and have been reported to be of use in the diagnosis of VZV meningoencephalitis from CSF specimens. The isolation of VZV from fibroblast culture is considered the definitive method for diagnosing VZV infections. However, virus isolation is rarely carried out because of the length of time required for a result to be available (up to 15 days), and the low sensitivity of 40% to 50%.

The present inventors have demonstrated, for the first time that VZV is both shed and detectable in the saliva of subjects. Their findings demonstrate the usefulness of saliva for the detection of virus in patients with VZV as well as herpes zoster. VZV DNA was present in the saliva of every patient with zoster early during disease. Interestingly, there were a few instances when VZV DNA was found in the saliva of patients with herpes zoster after pain had disappeared, and once when radicular pain preceded rash. Given that there have been multiple reports of virologically confirmed VZV-induced neurological disease without any history of zoster rash—including myelitis, cerebellar ataxia, meningoencephalitis, VZV vasculopathy, and zoster sine herpete—it will be important to determine whether VZV DNA, VZV proteins, or antibodies against VZV can be detected in the saliva of such patients. To date, definitive virological confirmation has required blood and cerebrospinal fluid examination for VZV DNA and anti-VZV IgG. Thus, the ability to detect VZV in a non-invasive fashion is a major advance.

The inventors have extended this observation by developing a device for the rapid, accurate and efficient testing for VZV in saliva. The device contains a reagent chamber, for example a syringe, having a first antibody conjugated to a microbead, and a second antibody conjugated to a detectable marker, a detection chamber comprising a capture agent for binding the microbead; and a disposal reservoir. The reagent chamber and detection chamber are designed to permit transfer of a fluid sample (i.e., saliva) therebetween, as are the detection chamber and the disposal reservoir. The device may optionally be fitted with a mouthpiece that attaches to the reagent chamber to facilitate obtaining of the saliva sample. This device will permit relatively unskilled healthcare workers to quickly and efficiently obtain samples that permit the rapid detection of VZV in subjects. It can also be used for detection of additional embodiments, including others viruses, bacteria, fungi, toxins, and environmental contaminants that are present in either a patient sample or in an environmental sample.

### I. VARICELLA ZOSTER VIRUS

A. Background

Varicella zoster virus (VZV) is one of eight herpes viruses known to infect humans (and other vertebrates). It commonly causes chicken-pox in children, and both shingles and postherpetic neuralgia in adults. VZV is known by many names, includings: chickenpox virus, varicella virus, zoster virus, and human herpes virus type 3 (HHV-3).

Primary VZV infection results in chickenpox (varicella), which may rarely result in complications including encephalitis or pneumonia. The infection affects mucosa and skin (scalp, face, trunk, proximal limbs). The symptoms include malaise, fever, pharyngitis, pruritis, nausea, headaches, and rash. The lesions are characterised by erythematous macules, papules (12-14 hr), vesicles, pustules, crust and then scarring (sometimes). The recovery takes about 2-3 weeks.

Even when clinical symptoms of chickenpox have resolved, VZV remains dormant in the nervous system of the infected person (virus latency), in the trigeminal and dorsal root ganglia. In about 10-20% of cases, VZV reactivates later in life producing a disease known as herpes zoster or shingles. Serious complications of shingles include postherpetic neuralgia, zoster multiplex, myelitis, herpes ophthalmicus, or zoster sine herpete.

VZV is closely related to the herpes simplex viruses (HSV), sharing much genome homology. The known envelope glycoproteins (gB, gC, gE, gH, gI, gK, gL) correspond with those in HSV, however there is no equivalent of HSV gD. VZV also fails to produce the LAT (latency-associated transcripts) that play an important role in establishing HSV latency (herpes simplex virus).

VZV virons are spherical and 150-200 nm in diameter. Their lipid envelope encloses the nucleocapsid of 162 capsomeres arranged in a icosahedral form. Its DNA is a single, linear, double-stranded molecule, 125,000 nt long. The capsid is surrounded by a number of loosely associated proteins known collectively as the tegument; many of these proteins play critical roles in initiating the process of virus reproduction in the infected cell. The tegument is in turn covered by a lipid envelope studded with glycoproteins that are displayed on the exterior of the virion.

The virus is very susceptible to disinfectants, notably sodium hypochlorite. Within the human body it can be treated by a number of drugs and therapeutic agents including aciclovir, zoster-immune globulin (ZIG), and vidarabine.

Varicella is normally a mild disease in immunocompetent individuals and no specific treatment is normally required. Varicella in immunocompromised patients can be a serious and potentially fatal disease. Acyclovir is now the drug of choice (VZV is not as susceptible to acyclovir as is HSV and requires 10-fold higher concentration of the drug for effective inhibition) for the treatment of varicella in the immunocompromised and also in normal patients with VZV pneumonia. A compound known as 882C had specific activity against VZV but without any significant activity against HSV or CMV. Specificity is achieved by the requirement of the drug for VZV thymidine kinase in order to be converted to the monophosphate and the diphosphate form.

B. Traditional Detection/Diagnosis

The clinical presentations of varicella or zoster are so characteristic that laboratory confirmation is rarely required. Laboratory diagnosis is required only for atypical presentations, particularly in the immunocompromised, and for distinguishing between HSV infection and herpes zoster.

Cytology involves examining smears of scrapings of the base of the lesions will reveal characteristic multinucleate giant cells, also known as Tzanck cells. However this technique will not distinguish between HSV and VZV infection. Electron microscopy permits observation of herpesvirus particles in fluid taken from the early vesicles of either varicella or zoster. This technique cannot distinguish between HSV and VZV. Immunofluorescence cytology uses smears of the base of lesions can be examined by immunofluorescence cytology, as in the case for HSV. This technique is more sensitive than EM, but is more labor intensive and requires greater technical expertise. Molecular methods, such as PCR assays for VZV, are available and have been reported to be of use in the diagnosis of VZV meningoencephalitis from CSF specimens.

Virus isolation remains the definitive method for diagnosing VZV infections. Human fibroblasts are used in most laboratories. Vesicle fluid and scrapings form the base of fresh lesions are the most suitable specimens. Virus can rarely be recovered from crusted lesions. Biopsy material can also be cultured. The CPE produced by VZV is so characteristic that most laboratories do not undertake further identification of the isolates. Immunofluorescence of the cell sheet by monoclonal antibodies is the method of choice for identification. Virus isolation for VZV is rarely carried out because of the long length of time required for a result to be available.

The most important use for serology is the determination of immune status before the administration of prophylactic therapy. Serological diagnosis of primary varicella infection can be reliably carried out using paired acute and convalescent sera. However, this is less reliable in the case of herpes zoster where there is specific antibodies present already. Therefore it is essential to obtain the first sample as soon as possible after the onset of the rash in order to demonstrate a rising titre. The sharing of antigens between HSV and VZV can make the interpretation of results very difficult. Where possible, the serological diagnosis should be backed up by virus isolation.

CFT is the most frequently used test. It is perfectly adequate for the diagnostic purposes but is too insensitive for immune status screening. Immunofluorescence is appreciably more sensitive than CFT and is used for the determination of immune status in many laboratories. RIA and EIA are the most sensitive methods available and therefore are the preferred method for determining immune status. EIA are rapidly becoming the most commonly used method for the determination of immune status. VZV IgM can be determined by IF and capture RIA or EIA. VZV IgM is produced in primary varicella and herpes zoster and thus it is not possible to distinguish between the two. However, the VZV IgM tests are likely to prove invaluable in determining the nature of congenital varicella infections

### II. DETECTION METHODS

The present invention permits, for the first time, the detection of VZV infection, primary or recurrent, in the saliva of subjects that do not have Ramsay Hunt syndrome (geniculate zoster). The detection of VZV particles may rely on immunological methods or those that detect VZV nucleic acids. Such methodologies are described below.

A. Immunodiagnostics

According to the inventors' findings, VZV particles can be detected in saliva. As a consequence, testing for the presence of VZV in fluids permits early, rapid, and inexpensive identification of the disease, and therefore early detection, with the goal of earlier intervention and limiting of circumstantially life-threatening disease. In certain embodiments, the methods are adapted for

The methods of the present invention rely, in one aspect, on immunologic detection using antibodies that bind to antigens on the surface of the VZV particle. Such antibodies are available from commercial sources, such as Santa Cruz Biotechnology, having a variety of specificities.

TABLE 1
VZV Antibodies

| PRODUCT | CATALOG | ISO- | | | SPE- |
|---|---|---|---|---|---|
| NAME | # | TYPE | EPITOPE | APPLNS | CIES |
| VZV gB | sc-56993 | mouse | FL (v) | IF | VZV |
| (10G6) | | IgG₁ | | | |
| Antibody | | | | | |
| VZV gE (vN- | sc-17549 | goat | N- | WB, | VZV |
| 20) Antibody | | IgG | terminus | ELISA | |
| | | | (VZV) | | |
| VZV gE | sc-56994 | mouse | FL (v) | WB, IF | VZV |
| (13B1) | | IgG₁ | | | |
| Antibody | | | | | |
| VZV gE (9C8) | sc-56995 | mouse | FL (v) | WB, IF | VZV |
| Antibody | | IgG₁ | | | |
| VZV gH (6A6) | sc-56996 | mouse | FL (v) | IF | VZV |
| Antibody | | IgG₁ | | | |
| VZV gI (8C4) | sc-56997 | mouse | FL (v) | WB, IF | VZV |
| Antibody | | IgG₁ | | | |
| VZV gI (vC- | sc-17510 | goat | C- | WB, | VZV |
| 20) Antibody | | IgG | terminus | ELISA | |
| VZV gI (vH- | sc-17509 | goat | internal | WB, | VZV, |
| 20) Antibody | | IgG | | ELISA | HHV-3 |
| VZV ie62 (vC- | sc-17525 | goat | C- | WB, | VZV, |
| 20) Antibody | | IgG | terminus | ELISA | HHV-3 |
| VZV | sc-56998 | mouse | FL (v) | IF | VZV |
| Immediate | | IgG₁ | | | |
| Early Protein | | | | | |
| (8B11) | | | | | |
| Antibody | | | | | |
| VZV Major | sc-56999 | mouse | FL (v) | IF | VZV |
| Capsid Protein | | IgG₁ | | | |
| (3H2) | | | | | |
| Antibody | | | | | |
| VZV NC | sc-58082 | mouse | FL (v) | IF | VZV |
| (0371) | | IgG₁ | | | |
| Antibody | | | | | |
| VZV thymidine | sc-17554 | goat | C- | WB, | VZV, |
| kinase (vC-20) | | IgG | terminus | ELISA | HHV-3 |
| Antibody | | | | | |

Alternatively, antibodies to VZV may be prepared using standard technologies, as explained below.

1. Preparing Antibodies

Methods for the production of antibodies are well known in the art, as described in see, e.g., Harlow and Lane, 1988; U.S. Pat. No. 4,196,265. The methods for generating monoclonal antibodies (MAbs) generally begin along the same lines as those for preparing polyclonal antibodies. The first step for both these methods is immunization of an appropriate host. As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas are then serially diluted and cloned into individual antibody-producing cell lines, which clones can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Various methods may be employed for the cloning an expression of human light and heavy chain sequences. Wardemann et al. (2003) and Takekoshi et al. (2001), both of which disclose such techniques, are hereby incorporated by reference.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobin preparations; and U.S. Pat. No. 4,867,973 which describes antibody-therapeutic agent conjugates.

2. Antibody Conjugation

Antibodies may be conjugated to supports (e.g., column matrices, bead) or labels using a variety of different reagents and methods. These include those described in U.S. Pat. Nos. 5,892,628 and 5,872,261, which describe sulfosuccinimidyl-6-(biotinamido) hexanoate linkers. Other approaches include NHS-ester reaction, maleimide reaction, imidoester reaction, active halogen, EDC coupling, pyridyl disulfide reaction, sulfo-NHS and enhanced EDC reactions.

3. Immunologic Assays

Antibodies of the present invention can be used in identifying VZV particles in saliva through techniques such as RIAs, ELISAs and Western blotting. This provides rapid, early, simple and cost-effective detection of infection.

In one aspect, an ELISA assay is contemplated. For example, antibodies to VZV antigens may be immobilized onto a selected surface, for example, a surface such as a microtiter well, a membrane, a filter, a bead or a dipstick. After washing to remove incompletely adsorbed material, it is desirable to bind or coat the surface with a non-specific agent that is known to be antigenically neutral with regard to the test sample, e.g., bovine serum albumin (BSA), casein or solutions of powdered milk. This allows for blocking of non-specific adsorption sites on the immobilizing surface and thus reduces the background caused by non-specific binding of antibody to antigen on the surface.

After binding of antibody to the surface and coating, the surface is exposed to urine, prostate fluid or semen. Following formation of specific immunocomplexes between antigens in the urine, prostate fluid or semen and the antibody, and subsequent washing, the occurrence and even amount of immunocomplex formation may be determined by subjecting the same to a second antibody having specificity for the antigen. Appropriate conditions preferably include diluting the sample with diluents such as BSA, bovine gamma globulin (BGG) and phosphate buffered saline (PBS)/Tween®. These added agents also tend to assist in the reduction of non-specific background. The detecting antibody is then allowed to incubate for from about 2 to about 4 hr, at temperatures preferably on the order of about 25° to about 27° C. Following incubation, the surface is washed so as to remove non-immunocomplexed material. A preferred washing procedure includes washing with a solution such as PBS/Tween®, or borate buffer.

To provide a detecting means, the second antibody will preferably have an associated label, e.g., an enzyme that will generate a color development upon incubating with an appropriate chromogenic substrate. Thus, for example, one will desire to contact and incubate the second antibody for a period of time and under conditions which favor the development of immunocomplex formation (e.g., incubation for 2 hr at room temperature in a PBS-containing solution such as PBS/Tween®).

After incubation with the second antibody, and subsequent to washing to remove unbound material, the amount of label is quantified (e.g., by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label). Quantitation is then achieved by measuring the label, e.g., degree of color generation, e.g., using a visible spectrum spectrophotometer. Other potential labels include radiolabels, fluorescent labels, dyes and chemilluminescent molecules (e.g., luciferase).

In one particular embodiment, a primary antibody may be conjugated to a bead for capture of the target (VZV virus) and a secondary antibody directed against the primary antibody (to VZV) is used and is conjugated to the detection reagent. Another embodiment includes capture antibody conjugated to a capture bead and targeted to epitope 1, and a detection antibody conjugated to a detection reagent and targeted to epitope 2. A third embodiment would be an antibody is biotinylated, and after binding to the target (i.e., VZV), a micro-bead bound to streptavidin is used to capture the target. Detection is then achieved with an anti-biotin antibody conjugated to a detection reagent.

The One-Step Antibody Biotinylation Kit (Miltenyi Biotec) has been developed for the biotinylation of monoclonal antibodies for use in magnetic cell separation (with MACS® Technology) as well as fluorescent cell analysis. Once biotinylated, antibodies can be directly used to tag target cells. Subsequently, target cells can either be magnetically labeled using MACS® Anti-Biotin MicroBeads (#130-090-485) or fluorescently stained with a FITC-, PE-, or APC-conjugated Anti-Biotin antibody. After labeling with Anti-Biotin MicroBeads, target cells can be enriched according to the protocol in the Anti-Biotin MicroBeads data sheet. The antibody to be biotinylated must be purified from azide, serum components, and other NH₂-containing molecules prior to biotinylation. The antibody should be prepared at a concentration of 100 μg/mL in PBS. A maximum of 10 μg of antibody can be labeled per well.

4. Dipstick Technology

U.S. Pat. No. 4,366,241, and Zuk, EP-A 0 143 574 describe migration type assays in which a membrane is impregnated with the reagents needed to perform the assay. An analyte detection zone is provided in which labeled analyte is bound and assay indicia is read.

U.S. Pat. No. 4,770,853, WO 88/08534, and EP-A 0 299 428 describe migration assay devices which incorporate within them reagents which have been attached to colored direct labels, thereby permitting visible detection of the assay results without addition of further substances.

U.S. Pat. No. 4,632,901, disclose a flow-through type immunoassay device comprising antibody (specific to a target antigen analyte) bound to a porous membrane or filter to which is added a liquid sample. As the liquid flows through the membrane, target analyte binds to the antibody. The addition of sample is followed by addition of labeled antibody. The visual detection of labeled antibody provides an indication of the presence of target antigen analyte in the sample.

EP-A 0 125 118, disclose a sandwich type dipstick immunoassay in which immunochemical components such as antibodies are bound to a solid phase. The assay device is “dipped” for incubation into a sample suspected of containing unknown antigen analyte. Enzyme-labeled antibody is then added, either simultaneously or after an incubation period. The device next is washed and then inserted into a second solution containing a substrate for the enzyme. The enzyme-label, if present, interacts with the substrate, causing the formation of colored products which either deposit as a precipitate onto the solid phase or produce a visible color change in the substrate solution.

EP-A 0 282 192, disclose a dipstick device for use in competition type assays. U.S. Pat. No. 4,313,734 describes the use of gold sol particles as a direct label in a dipstick device. U.S. Pat. No. 4,786,589 describes a dipstick immunoassay device in which the antibodies have been labeled with formazan.

U.S. Pat. No. 5,656,448 pertains to dipstick immunoassay devices comprising a base member and a single, combined sample contact zone and test zone, wherein the test zone incorporates the use of symbols to detect analytes in a sample of biological fluid. A first immunological component, an anti-immunoglobulin capable of binding to an enzyme-labeled antibody, is immobilized in a control indicator portion. A second immunological component, capable of specifically binding to a target analyte which is bound to the enzyme-labeled antibody to form a sandwich complex, is immobilized in a test indicia portion. The enzyme-labeled antibody produces a visual color differential between a control indicia portion and a non-indicia portion in the test zone upon contact with a substrate. The device additionally includes a first polyol and a color differential enhancing component selected from the group consisting of an inhibitor to the enzyme and a competitive secondary substrate for the enzyme distributed throughout the non-indicia portion of the test zone.

5. Kits and Devices

In still further embodiments, the present invention concerns immunodetection kits for use with the immunodetection methods described above. However, the device can be used to detect any substance that can be identified using antibody or nucleic acid labeling and detection technology. These targets include, but are not limited to, numerous biologically hazardous materials including agents of bioterrorism (ricin, botulism toxin). The device is a closed system, minimizing exposure of body fluids to medical staff, making it an excellent tool for the detection of infectious disease. It also is portable, compact, light weight and self-contained making it ideal for use in remote locations including military field operations (e.g., bioterrorism detection from environmental or biological samples) as well as remote rural locations (i.e., screening rural populations or animal populations, for Ebola/HIV virus, cholera, smallpox, rabies, “mad cow” disease). The device is flexible in that it can be used with virtually any body fluid or with liquid extracts of tissue samples or even environmental samples. The only requirement is that the sample is liquid of can be suspended in a solution that can be passed through the column. The device even has ability to function under conditions of zero gravity, suitable for use in space.

The device may be readily assembled from commercially available products. Materials used for the components that make up this device include ABS, polypropylene, polycarbonate PVC and silicone, and combinations thereof.

i. Syringes

A syringe is simple piston pump consisting of a plunger that fits tightly in a tube. The plunger can be pulled and pushed along inside a cylindrical tube (the barrel), allowing the syringe to take in and expel a liquid or gas through an orifice at the open end of the tube. The open end of the syringe may be connected with other devices, such as a hypodermic needle, a nozzle, or tubing to help direct the flow into and out of the barrel. This is often accomplished by incorporate of a Luer Lok, which permits connection of the syringe to other devices.

The barrel of a syringe is made of plastic or glass, and usually has graduated marks indicating the volume of fluid in the syringe, and is nearly always transparent. Glass syringes may be sterilized in an autoclave. However, most modern syringes are plastic with a rubber piston, because this type seals much better between the piston and the barrel and because they are cheap enough to dispose of after being used only once, reducing the risk of spreading blood-borne diseases.

Examples include Becton-Dickson Luer Lok 10 ml or 30 cc syringe and Qosina Corp. 10 ml polycarbonate syringe

ii. Columns

Columns and microcolumsn permit purification of compounds from mixtures, including patient samples. The classical preparative chromatography column is a glass tube with a tap at the bottom. Eluent is slowly passed through the column to advance the sample material. The individual components are retained by the stationary phase (depending on the type of column, this may be by affinity) as they pass through the column with the eluent. The stationary phase or adsorbent in column chromatography is a solid. The mobile phase or eluent is either a pure sample or sample+solvent. One suitable example is the autoMACS Column from Miltenyi Biotec.

iii. Collection/Disposal Bags

Collection or disposal bags can be of virtually any suitable material (rubber, latex, polyvinyl), an include commerically available urine collection (Bard Center Entry urine drainage bags) and i.v. bags (Braun 150 ml i.v. bags).

iv. Exemplary VZV Kit

The kits will include antibodies to VZV, and may contain other reagents as well. The immunodetection kits will thus comprise, in suitable container means, a first antibody that binds to VZV, and optionally a second and distinct antibody to VZV. Also included may be an antibody that binds to the constant region of the first antibody, the antibody being labeled.

In certain embodiments, the antibody to VZV may be pre-bound to a solid support, such as a column matrix, a microtitre plate, a filter, a membrane, a bead or a dipstick. The immunodetection reagents of the kit may take any one of a variety of forms, including antibodies to VZV containing detectable labels. As noted above, a number of exemplary labels are known in the art and all such labels may be employed in connection with the present invention.

The kits may further comprise a suitably aliquoted composition of VZV antigen, whether labeled or unlabeled, as may be used to prepare a standard curve for a detection assay. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present invention will also typically include a means for containing the antibody, antigen, and any other reagent containers in close confinement for commercial sale. Such containers may include injection or blow-molded plastic containers into which the desired vials are retained.

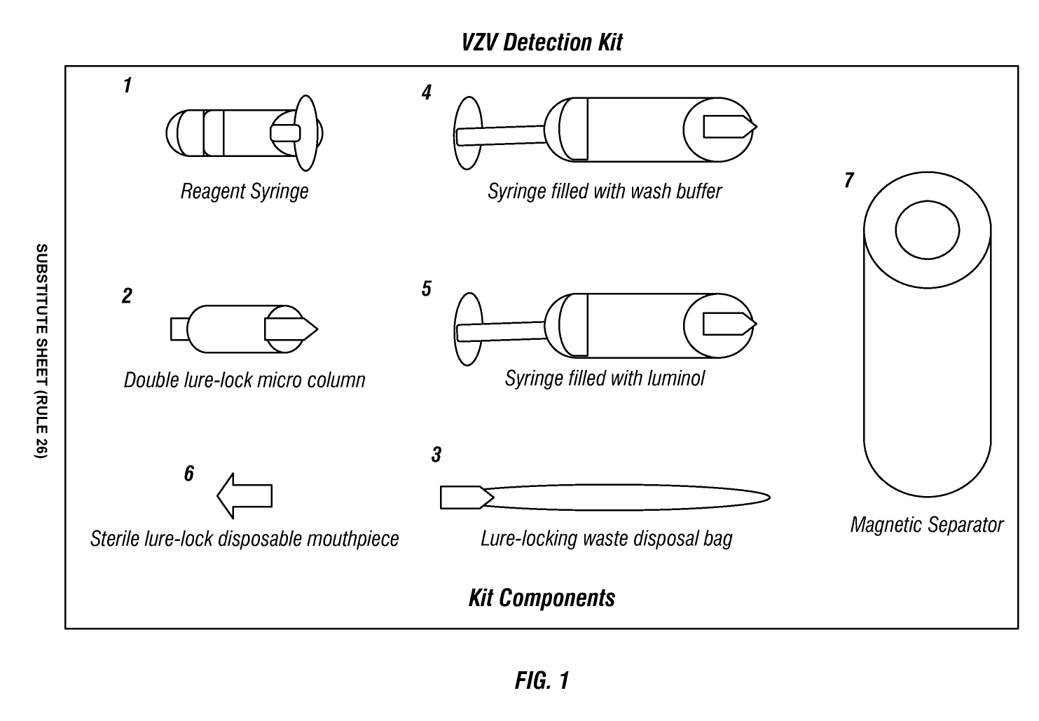

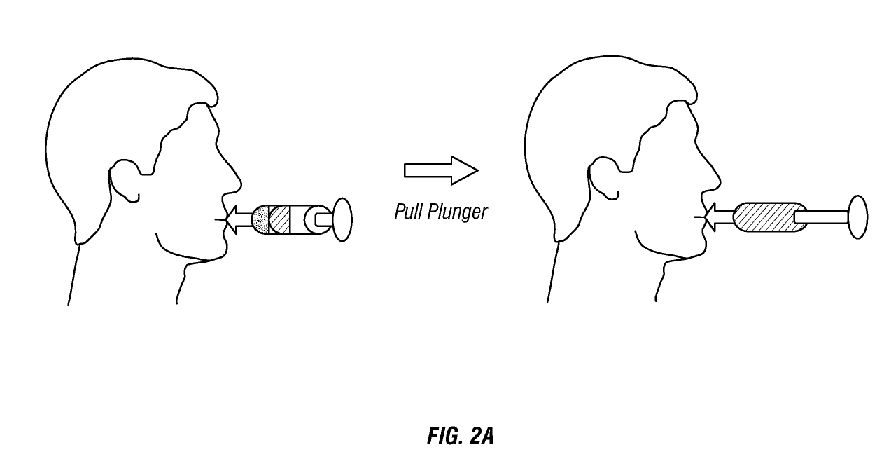

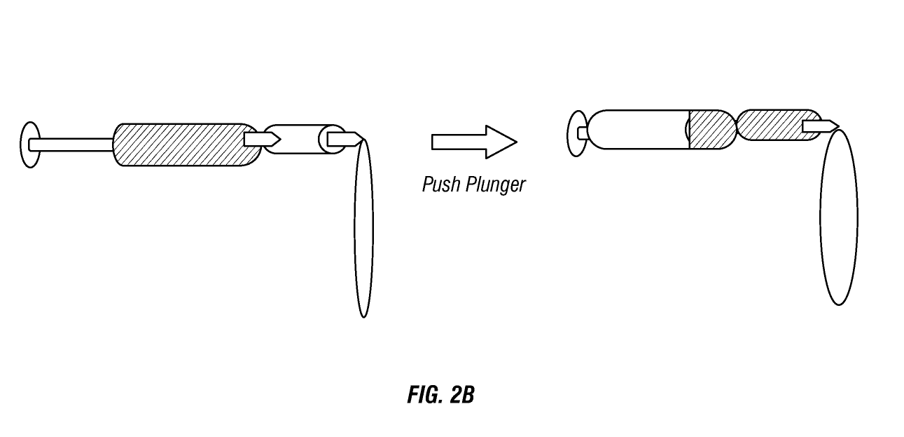

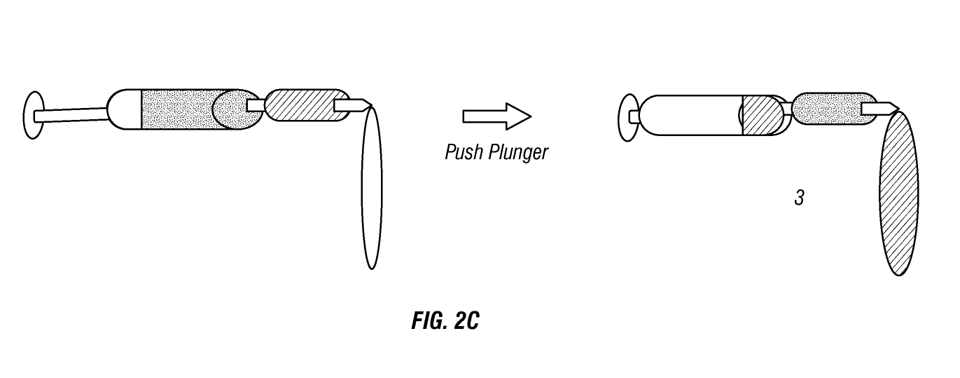

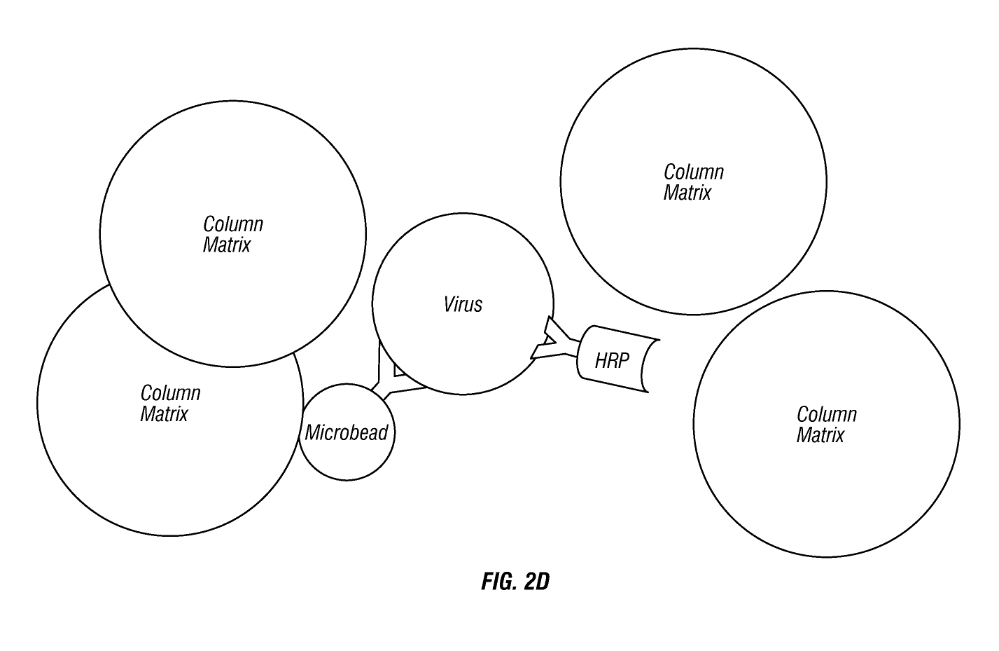

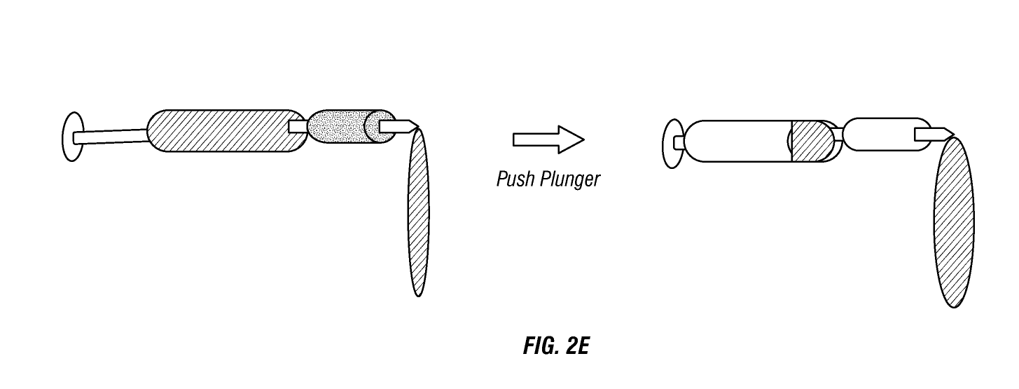

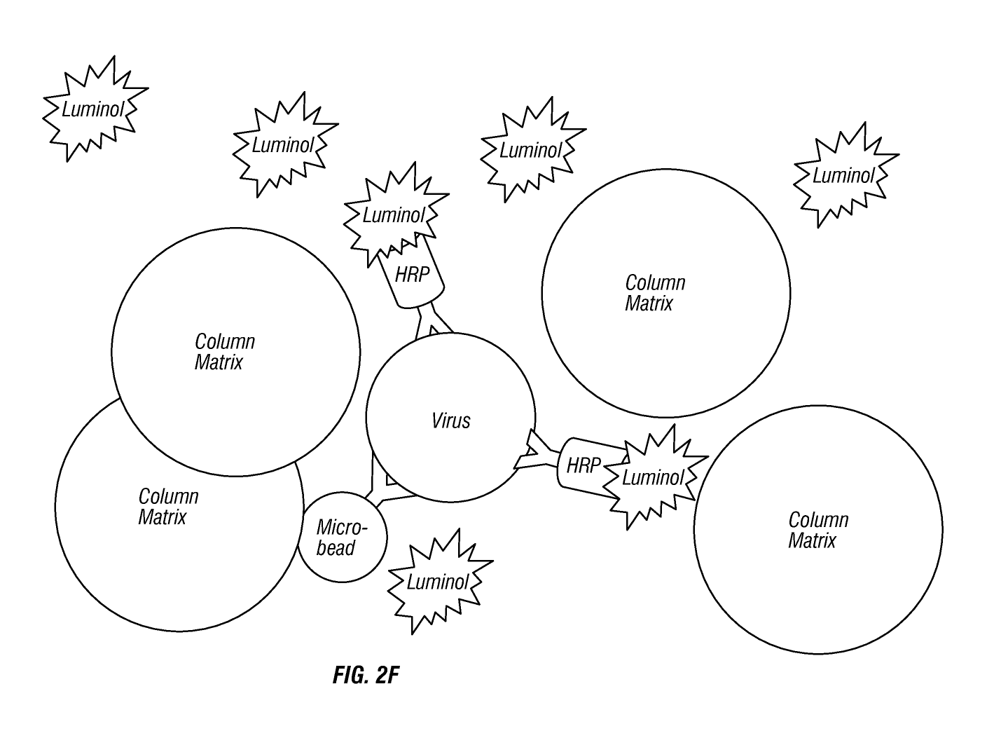

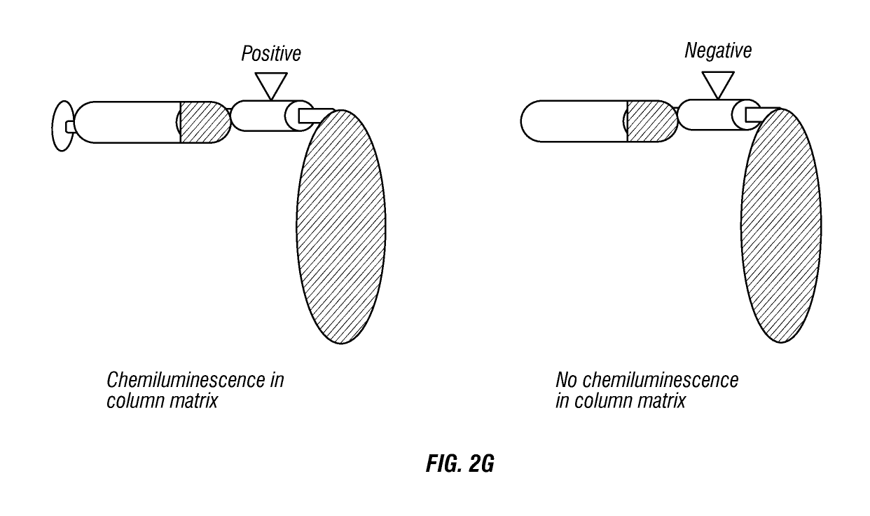

In a particular embodiment, a device that permits rapid, safe and simple obtention of saliva samples, and testing thereof, is provided. As shown in FIG. 1, a detection device may have seven different components. The components include a reagent chamber/syringe 1, which will contain two types of antibodies to VZV—one that permits capture on support (e.g., a bead), and one that permits subsequent detection of support-bound VZV. A reaction chamber 2, e.g., a micro-column, is included that attaches to the reagent syringe and permits transfer of the contents (saliva+antibody bound virus) from the reagent syringe into the reaction chamber. A waste disposal chamber/bag 3 can be attached to the other end of the reaction chamber to capture any saliva that follows through the reaction chamber. A wash syringe 4 filled with wash buffer may be connected to the reaction chamber, permitting transfer of the wash buffer into the reaction chamber and through to the disposal bag 3. A detection syringe 5, filled with a detection agent (e.g., Luminol™), can then be attached to the reaction chamber 2. Transfer of the reaction agent into the reaction chamber 2 and through into the wash bag 3 is facilitated. A mouthpiece 6 can be attached to the reagent syringe 1 to assist in obtaining the sample. A magnetic separator 7 may also be included to assist in fixing magnetic beads that have been transferred into the reaction chamber 3. In FIGS. 2A-G, a sequence of steps using the device of FIG. 1 is shown.

v. Other Targets

The detection device, as discussed above, may be utilized for detection of a variety of other agents. These include bioterrorism agents, such as Anthrax (Bacillus anthracis), Arenaviruses, Botulism (Clostridium botulinum toxin), Brucella species (brucellosis), Brucellosis (Brucella species), Burkholderia mallei (glanders), Burkholderia pseudomallei (melioidosis), Chlamydia psittaci (psittacosis), Cholera (Vibrio cholerae), Clostridium perfringens (Epsilon toxin), Coxiella burnetii (Q fever), Ebola virus hemorrhagic fever, E. coli O157:H7 (Escherichia coli), Nipah virus, Hantavirus, Epsilon toxin of Clostridium perfringens, food safety threats, (e.g., Salmonella species, Escherichia coli O157:H7, Shigella), Francisella tularensis (tularemia), Glanders (Burkholderia mallei), Lassa fever, Marburg virus hemorrhagic fever, Melioidosis (Burkholderia pseudomallei), Plague (Yersinia pestis), Ricin toxin from Ricinus communis (castor beans), Rickettsia prowazekii (typhus fever), Salmonella species (salmonellosis), Salmonella Typhi (typhoid fever), Shigella (shigellosis), Smallpox (variola major), Staphylococcal enterotoxin B, Tularemia (Francisella tularensis), Typhoid fever (Salmonella typhi), Variola major (smallpox), viral encephalitis (alphaviruses, e.g., Venezuelan equine encephalitis, eastern equine encephalitis, western equine encephalitis), viral hemorrhagic fever, (filoviruses, e.g., Ebola, Marburg) and arenaviruses (e.g., Lassa, Machupo), and water safety threats (e.g., Vibrio cholerae, Cryptosporidium parvum).

Human viruses that may be assessed include flaviviruses, hepadnaviruses, herpesviruses, poxviruses, arenaviruses, bunyaviruses, reoviruses, retroviruses, filoviruses, rhabdoviruses, coronaviruses, togaviruses, astroviruses, picornaviruses, parvoviruses, orthomyxoviruses, paramyxoviruses, papovaviruses and adenoviruses. Specific viruses include HIV, human papilloma viruses, Epstein Barr virus, cytomegalovirus, respiratory syncytial virus, West Nile virus, and influenza virus.

Bacteria that may be assessed include Acinetobacter baumanii (Family Moraxellaceae), Actinobacillus spp. (Family Pasteurellaceae), Actinomycetes (actinomycetes, streptomycetes), Actinomyces spp. (Actinomyces israelii, Actinomyces naeslundii, Actinomyces spp.), Aeromonas spp. (Family Aeromonadaceae), Aeromonas hydrophila, Aeromonas veronii biovar sobria (Aeromonas sobria), Aeromonas caviae, Peptostreptococcus spp., Streptococcus spp., Veillonella spp., Actinomyces israelii, Actinomyces naeslundii, Mobiluncus spp., Propionibacterium acnes, Lactobacillus spp., Eubacterium spp., Bifidobacterium spp., Bacteroides spp., Prevotella spp., Porphyromonas spp., Fusobacterium spp., Bacillus spp., Bacillus anthracis, Bacillus cereus, Bacillus subtilis, Bacillus thuringiensis, Bacillus stearothermophilus, Bacteroides fragilis, Bordetella spp., Bordetella pertussis, Bordetella parapertussis, Bordetella bronchiseptica, Borrelia recurrentis, Borrelia burgdorferi, Brucella spp., Brucella abortus, Brucella canis, Brucella melintensis, Brucella suis, Burkholderia pseudomallei, Burkholderia cepacia, Campylobacter jejuni, Campylobacter coli, Campylobacter lari, Campylobacter fetus, Citrobacter spp. (Family Enterobacteriaceae), Clostridium spp., Clostridium perfringens, Clostridium difficile, Clostridium botulinum, Corneybacterium spp., Corynebacterium diphtheriae, Corynebacterium jeikeum, Corynebacterium urealyticum, Edwardsiella tarda (Family Enterobacteriaceae), Enterobacter spp. (Family Enterobacteriaceae), Family Enterobacteriaceae, Citrobacter freundii, Citrobacter diversus, Enterobacter spp., Enterobacter aerogenes, Enterobacter agglomerans, Enterobacter cloacae, Escherichia coli, ETEC, EIEC, EPEC, EHEC, EaggEC, UPEC, Klebsiella spp. (Klebsiella pneumoniae; Klebsiella oxytoca), Morganella morganii, Proteus spp. (Proteus mirabilis; Proteus vulgaris), Providencia spp. (Providencia alcalifaciens; Providencia rettgeri; Providencia stuartii), Salmonella spp. (Salmonella enterica; Salmonella typhi; Salmonella paratyphi; Salmonella enteritidis; Salmonella cholerasuis; Salmonella typhimurium), Serratia spp. (Serratia marcesans, Serratia liquifaciens), Shigella spp. (Shigella dysenteriae, Shigella flexneri, Shigella boydii, Shigella sonnei), Yersinia spp. (Yersinia enterocolitica, Yersinia pestis, Yersinia pseudotuberculosis), Enterococcus spp. (gamma hemolytic, occasionally alpha or beta; formerly classified as Group D streptococci; Enterococcus faecalis; Enterococcus faecium), Erysipelothrix rhusopathiae, Francisella tularensis, Haemophilus spp. (Family Pasteurellaceae; Haemophilus influenzae; Haemophilus ducreyi; Haemophilus aegyptius; Haemophilus parainfluenzae; Haemophilus haemolyticus; Haemophilus parahaemolyticus), Helicobacter spp. (Helicobacter pylori; Helicobacter cinaedi; Helicobacter fennelliae), Legionella pneumophila, Leptospira interrogans (Order Spirochaetales; Family Leptospiraceae), Listeria monocytogenes, Micrococcus spp. (Family Micrococcaceae), Moraxella catarrhalis (Family Moraxellaceae or Family Neisseriaceae), Morganella spp. (Family Enterobacteriaceae), Mycobacterium spp. (Family Mycobacteriaceae; Mycobacterium leprae; Mycobacterium tuberculosis), Nocardia spp. (Family Nocardiaceae; Nocardia asteroides; Nocardia brasiliensis), Neisseria spp. (Family Neisseriaceae; Neisseria gonorrhoeae; Neisseria meningitidis); Pasteurella multocida (Family Pasteurellaceae), Plesiomonas shigelloides (Family Plesiomonadaceae), Propionibacterium acnes, Pseudomonas aeruginosa (Family Pseudomonadaceae), Rhodococcus spp. (actinomycetes with mycolic acids, Family Nocardiaceae), Staphylococcus spp. (Family Micrococcaceae; catalase positive), Staphylococcus aureus (coagulase-positive), Staphylococcus epidermidis (coagulase-negative), Staphylococcus saprophyticus (coagulase-negative), Stenotrophomonas maltophilia, Streptococcus pneumoniae (no group specific carbohydrate; alpha hemolytic); Streptococcus spp. (Family Streptococcaceae) (catalase negative), Group A streptococci (beta hemolytic; Streptococcus pyogenes), Group B streptococci (beta hemolytic, occasionally alpha or gamma; Streptococcus agalactiae), Group C streptococci (beta hemolytic, occasionally alpha or gamma; Streptococcus anginosus; Streptococcus equismilis), Group D streptococci (alpha or gamma hemolytic, occasionally beta; Streptococcus bovis), Group F streptococci (beta hemolytic; Streptococcus anginosus); Group G streptococci (beta hemolytic; Streptococcus anginosus); Viridans streptococci (no group specific carbohydrate) (alpha or gamma hemolytic), Streptococcus mutans, Streptococcus salivarius group, Streptococcus sanguis group, Streptococcus mitis group, Streptomyces spp. (actinomycetes, streptomycetes); Treponema spp. (Order Spirochaetales; Family Spirochaetaceae; Treponema pallidum, ssp. pallidum, Treponema pallidum ssp. endemicum, Treponema pallidum ssp. Pertenue, Treponema carateum, Vibrio spp. (Family Vibrionaceae), Vibrio cholerae Serogroups O1 and O139, non-agglutinable vibrios (NAGs) or non-cholera vibrios (NCVs), Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii.

B. Nucleic Acid Based Detection

Certain embodiments of the present invention concern methods for detection of VZV nucleic acids in the saliva of subjects. These methods rely on the use of various nucleic acids, including amplification primers, oligonucleotide probes, and other nucleic acid elements involved in the analysis of genomic DNA. A “nucleic acid” as used herein will generally refer to a molecule (i.e., a strand) of DNA or RNA comprising a nucleobase—an adenine, guanine, cytosine or thymine (or uracil for RNA). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product.

In some embodiments, nucleic acids of the invention comprise or are complementary to all or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1165, 1200, 1300, 1400, 1500, 2000, 3000 or more contiguous nucleotides, or any range derivable therein, of a VZV nucleic acid. One of skill in the art knows how to design and use primers and probes for hybridization and amplification, including the limits of homology needed to implement primers and probes.

These definitions generally refer to a single-stranded molecule, but in specific embodiments will also encompass an additional strand that is partially, substantially, or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A nucleic acid may be purified on polyacrylamide gels, cesium chloride centrifugation gradients, chromatography columns or by any other means known to one of ordinary skill in the art (see for example, Sambrook et al. (2001), incorporated herein by reference). In some aspects, a nucleic acid is a pharmacologically acceptable nucleic acid. Pharmacologically acceptable compositions are known to those of skill in the art, and are described herein.

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

The present invention also encompasses a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

The target region(s) may be amplified using any oligonucleotide-directed amplification method, including but not limited to polymerase chain reaction (PCR) (U.S. Pat. No. 4,965,188), ligase chain reaction (LCR) (Barany et al., 1991; WO90/01069), and oligonucleotide ligation assay (OLA) (Landegren et al., 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., 1992).

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence. For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

A number of template dependent processes are available to amplify the nucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329,822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

### III. TREATMENT OF VZV INFECTION

A. Primary Infection (Chickenpox)

The most common method of dealing with primary VZV infection is to relieve their subject's symptoms. Because scratching blisters may cause them to become infected, this should be avoided. Calamine lotion and Aveeno® (oatmeal) baths may help relieve some of the itching. Aspirin or aspirin-containing products are not to be used to relieve fever in children, as the use of aspirin in children with chickenpox has been associated with development of Reye's syndrome (a severe disease affecting all organs, but most seriously affecting the liver and brain, that may cause death). Thus, non-aspirin medications such as acetaminophen (e.g., Tylenol®) are preferred.

Acyclovir, famcyclovir, or valacyclovir (medicines that work against herpesviruses) are recommended for persons who are more likely to develop serious disease, including persons with chronic skin or lung disease, otherwise healthy individuals 13 years of age or older, and persons receiving steroid therapy. However, only acyclovir is currently licensed for use in treating varicella. Persons whose immune systems have been weakened from disease or medication should contact their doctor immediately if they are exposed to or develop chickenpox. If a subject is pregnant and are either exposed to or develop chickenpox, she should immediately discuss prevention and treatment options with your doctor.

If a subject has a fever that lasts longer than 4 days or rises above 102° F., a health-care provider should be consulted. Also if any areas of the rash or any part of the body become very red, warm, or tender, or begin leaking pus (thick, discolored fluid), a health-care provider should be consulted as these symptoms may indicate a bacterial infection. Also, a doctor should be contacted immediately if the subject seems extremely ill, is difficult to wake up or appears confused, has difficulty walking, has a stiff neck, is vomiting repeatedly, has difficulty breathing, or has a severe cough.

Varicella zoster immune globulin (VZIG) can prevent or modify disease after exposure to someone with chickenpox. However, because it is costly and only provides temporary protection, VZIG is only recommended for persons at high risk of developing severe disease who are not eligible to receive chickenpox vaccine. These individuals include newborns whose mothers have chickenpox 5 days prior to 2 days after delivery, premature babies exposed to varicella in the first month of life, children with leukemia or lymphoma who have not been vaccinated, persons with cellular immunodeficiencies or other immune system problems, persons receiving medications, such as high-dose systemic steroids, that suppress the immune system, and women who are pregnant. VZIG should be administered as soon as possible, but no later than 96 hours, after exposure to chickenpox.

B. Recurrent Infection (Shingles)

There are several effective treatments for shingles, such as acyclovir (Zovirax®), valacyclovir (Valtrex®), or famciclovir (Famvir®). These antivirals can reduce the severity and duration of the rash if started early (within 72 hrs of the appearance of the rash). The addition of steroid drugs may have limited benefit in some cases, but studies have not conclusively confirmed the benefit of steroids in combination with all antiviral drugs. In addition to antiviral medication, pain medications may be needed for symptom control.

The affected area should be kept clean. Bathing is permitted, and the area can be cleansed with soap and water. Cool compresses and anti-itching lotions, such as calamine lotion, may also provide relief. An aluminum acetate solution (Burow's or Domeboro solution, available at your pharmacy) can be used to help dry up the blisters and oozing.

Generally, shingles heal well and problems are few. However, on occasion, the blisters can become infected with bacteria, causing cellulitis, a bacterial infection of the skin. If this occurs, the area will become reddened, warm, firm, and tender, red streaks may form around the wound. If any of these symptoms appear, a health-care professional should be consulted. Antibiotics can be used to treat these complications.

A more worrisome complication occurs when shingles affect the face, specifically the forehead and nose. In these cases, it is possible, although not likely, that shingles can affect the eye, leading to loss of vision. If shingles appear on the forehead or nose, eyes should be evaluated by a health-care professional.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Experimental Design. Fifty-four patients with herpes zoster were treated with valacyclovir. On treatment days 1, 8, and 15, pain was scored and saliva examined for varicella-zoster virus (VZV) DNA. VZV DNA was found in every patient the day treatment was started and later disappeared in 82%. There was a positive correlation between the presence of VZV DNA and pain and between VZV DNA copy number and pain (P<0.0005). VZV DNA was present in 1 patient before rash and in 4 after pain resolved and was not present in any of 6 subjects with chronic pain or in 14 healthy subjects. Analysis of human saliva has potential usefulness in the diagnosis of neurological disease produced by VZV without rash.

Varicella-zoster virus (VZV) DNA is present in the saliva of healthy astronauts and patients with Ramsay Hunt syndrome (geniculate zoster). The inventors hypothesized that a prospective analysis of patients with herpes zoster would detect VZV in saliva independent of zoster location.

Methods. All human-study protocols were approved by the Committee for the Protection of Human Subjects of the Lyndon B. Johnson Space Center (control subjects) and by the Institutional Review Board of the University of Texas Health Sciences Center (patients with herpes zoster). Informed consent was obtained from all subjects. The 54 patients with herpes zoster consisted of 29 women (21-82 years old) and 25 men (35-79 years old) (Table 2). All patients with herpes zoster were treated with oral valacyclovir (1 g 3 times daily) for 7 consecutive days. Pain was described by all patients on a scale of 0 (no pain) to 10 (worst pain) (Jacox et al., 1994). Six control subjects consisted of 5 women (24-64 years old) and 1 man (38 years old) with chronic pain due to malignancy or non-VZV inflammatory disease. Fourteen additional healthy control subjects consisted of 9 men and 5 women (34-70 years old). Three saliva samples were collected at weekly intervals from all 6 control subjects with chronic pain and from the 14 healthy control subjects.

Saliva samples from all subjects were obtained by having subjects suck on a cotton plug for 1-2 min, after which the saturated cotton was placed in a salivette tube (Sarstedt). The salivette tube was centrifuged at 1303 g for 10 min to collect saliva in the outer part of the salivette tube. Saliva was then concentrated with a Microsep 100K filtration unit (Filtron Technology) by centrifugation at 4552 g for 2 h. DNA was extracted with nonorganic extraction reagents (Qiagen). Microcarrier gel (Molecular Research Center) was added to facilitate DNA recovery. DNA was dissolved in 50 μL of nuclease-free water (Amresco). Quantitative real-time polymerase chain reaction (PCR) was performed in a TaqMan 7700 sequence detector (Applied Biosystems) using fluorescence-based simultaneous amplification and product detection.

Primers and probes specific for VZV, herpes simplex virus (HSV)-1 and GAPDH DNA sequences have been described elsewhere (Mehta et al., 2004; Cohrs et al., 2000). To avoid contamination from skin lesions, patients did not touch the salivette cotton roll while collecting saliva. Samples were collected on day 1 before antiviral therapy and again on days 8 and 15. Months after the study was under way and VZV was confirmed to be present in saliva of patients with herpes zoster, saliva from 2 patients (patients 11 and 19 in Table 2) were inoculated onto human fetal lung-cell fibroblasts and observed for cytopathic effect (CPE) (Cohrs et al., 2000). At the height of CPE, cells were analyzed by PCR with HSV-1-specific and VZV-specific primers (Mehta et al., 2004; Cohrs et al., 2000) and by immunohistochemistry for viral antigens.

Random-effects logistic regression was used to correlate pain with the presence of VZV DNA in the saliva of patients with herpes zoster (Diggle et al., 1995). Association between pain and the VZV DNA burden was determined using the Somers D test (Newson, 2006), a non-parametric analog to the regression coefficient in ordinary linear regression, which is related to forms of Kendall's τ (Kendall and Gibbons, 1990).

Results. All control subjects and patients with herpes zoster were seropositive for VZV (data not shown). Table 2 lists the level of pain reported by 54 patients with herpes zoster before and after treatment, as well as the VZV DNA copy number per milliliter of saliva, as detected by real-time PCR. For 50 of the 54 patients, follow-up data were available. Pain scores were available from all 54 patients on the day treatment was started (day 1), from 44 patients on day 8, and from 48 patients (not necessarily the same patients) on day 15. In 43 (86%) of 50 patients, pain decreased during the 14-day study period; in 2 patients (patients 33 and 45), pain transiently increased before decreasing. Ultimately, in 37 (74%) of the 50 patients, pain disappeared entirely during the 14-day study period. Three (6%) of the 50 patients (patients 9, 41, and 50) developed an increase in pain after it had disappeared. In 4 (8%) of 50 patients (patients 6, 9, 16, and 34), pain increased throughout the 14-day study period.

TABLE 2
Characteristics of patients with herpes zoster, pain scores, and salivary
varicella zoster virus (VZV) DNA burden during antiviral treatment

| | | | Days of | Days of | | | | | | |
|---|---|---|---|---|---|---|---|---|---|---|
| Patient | | | Pain Before | Rash Before | Day 1 (Start | | | | | |
| (Age in | Underlying | Zoster | Antiviral | Antiviral | of Treatment) | | Day 8 | | Day 15 | |
| Years/Sex) | Disease | Site^{a} | Treatment | Treatment | Pain | VZV^{b} | Pain | VZV^{b} | Pain | VZV^{b} |
| 1 (64/F) | None | V1 | 2 | 1 | 3 | 1.4 × 10⁷ | ND | ND | 0 | 0 |
| 2 (52/M) | Stomach cancer | V1 | 4 | 2 | 3 | 3.2 × 10⁴ | 0 | 0 | 0 | 0 |
| 3 (44/M) | None | V1 | 18 | 12 | 7 | 1.0 × 10⁵ | 3 | 9.3 × 10³ | 0 | 0 |
| 4 (48/F) | None | V1 | 1 | 1 | 2 | 1.8 × 10⁵ | 1 | 1.8 × 10³ | 1 | 1.8 × 10³ |
| 5 (59/F) | None | V1 | 1 | 1 | 1 | 6.3 × 10² | 0 | 0 | 0 | 0 |
| 6 (62/M) | Cancer | V1 | 14 | 7 | 5 | 2.5 × 10⁴ | 5 | 6.8 × 10⁴ | 7 | 4.7 × 10⁴ |
| 7 (41/M) | None | V1 | 6 | 2 | 6 | 1.0 × 10³ | 0 | 6.3 × 10² | 0 | 0 |
| 8 (82/F) | Infected finger | V1 | 7 | 2 | 4 | 5.5 × 10³ | 2 | 2.2 × 10³ | 0 | 0 |
| 9 (75/M) | Hypertension | C2 | 3 | 1 | 1 | 7.2 × 10⁴ | 0 | 3.4 × 10⁴ | 2 | 1.3 × 10³ |
| 10 (65/M) | Hypertension, | C2 | 7 | 3 | 8 | 5.0 × 10⁵ | 5 | 4.9 × 10² | 3 | 0 |
| | hypothyroidism | | | | | | | | | |
| 11 (79/M) | None | C2-C3 | 6 | 2 | 6 | 6.5 × 10³ | ND | ND | ND | ND |
| 12 (49/F) | None | C3 | 1 | 1 | 7 | 1.4 × 10⁷ | 2 | 7.5 × 10² | 0 | 0 |
| 13 (40/M) | Colon cancer | C3 | 3 | 7 | 3 | 1.2 × 10⁸ | 2 | 3.8 × 10³ | 2 | 3.5 × 10² |
| 14 (53/F) | Hepatitis | C3 | 6 | 1 | 6 | 8.0 × 10⁰ | ND | ND | 0 | 0 |
| 15 (44/F) | None | C3 | 3 | 1 | 7 | 5.8 × 10⁵ | 2 | 0 | 0 | 0 |
| 16 (48/M) | Malignant brain | C3 | 3 | 1 | 2 | 4.5 × 10¹ | 4 | 3.0 × 10² | 6 | 1.0 × 10⁵ |
| | tumor | | | | | | | | | |
| 17 (63/M) | Back surgery | C3-C4 | 14 | 10 | 4 | 6.5 × 10⁴ | 0 | 0 | 0 | 0 |
| 18 (26/F) | None | C4 | 21 | 3 | 6 | 8.2 × 10⁴ | 2 | 0 | 0 | 0 |
| 19 (26/F) | None | C4 | 12 | 5 | 4 | 4.5 × 10⁴ | ND | ND | ND | ND |
| 20 (47/F) | Cancer | 11 | 2 | 1 | 1 | 8.0 × 10¹ | 0 | 0 | 0 | 0 |
| 21 (58/F) | Lumbar disk | T2 | 14 | 4 | 5 | 2.0 × 10⁴ | 2 | 2.1 × 10⁷ | 0 | 0 |
| | disease | | | | | | | | | |
| 22 (53/F) | Asthma, anemia | T2-T3 | 13 | 3 | 6 | 6.6 × 10³ | ND | ND | ND | ND |
| 23 (66/F) | None | T3 | 5 | 1 | 3 | 6.9 × 10¹ | 0 | 0 | 0 | 0 |
| 24 (51/M) | None | T3-T4 | 7 | 6 | 2 | 4.2 × 10¹ | 0 | 0 | 0 | 0 |
| 25 (75/M) | None | T3-T4 | 4 | 2 | 2 | 4.3 × 10² | 0 | 0 | 0 | ND |
| 26 (39/F) | None | T4 | 5 | 3 | 4 | 2.3 × 10¹ | 0 | 0 | 0 | 0 |
| 27 48/M) | None | T4-T5 | 3 | 2 | 5 | 8.8 × 10⁴ | 0 | 3.1 × 10³ | 0 | 0 |
| 28 (30/F) | None | T5 | 4 | 3 | 6 | 4.4 × 10¹ | ND | ND | 0 | 0 |
| 29 (56/F) | Hyperlipidemia, | T5 | 5 | 3 | 3 | 6.5 × 10¹ | 0 | 0 | 0 | 0 |
| | kidney stone, | | | | | | | | | |
| | diabetes | | | | | | | | | |
| 30 (60/M) | None | T5 | 8 | 7 | 2 | 9.0 × 10⁰ | 0 | ND | 0 | 0 |
| 31 (65/M) | None | T5 | 3 | 1 | 8 | 1.9 × 10⁴ | 6 | 1.7 × 10⁵ | 0 | 0 |
| 32 (59/M) | None | T5 | 3 | 2 | 3 | 1.6 × 10¹ | 0 | 0 | 0 | 0 |
| 33 (62/M) | None | T5 | 12 | 5 | 1 | 1.6 × 10² | 4 | 0 | 2 | 0 |
| 34 (62/M) | None | T5-T6 | 3 | 2 | 5 | 1.2 × 10² | 6 | 4.8 × 10² | 6 | 4.5 × 10⁴ |
| 35 (58/F) | None | T6 | 4 | 2 | 2 | 1.3 × 10² | ND | ND | ND | ND |
| 36 (43/F) | None | T6 | 5 | 3 | 4 | 2.1 × 10¹ | 0 | 0 | ND | ND |
| 37 (61/M) | Hypertension, | T6 | 6 | 3 | 4 | 5.8 × 10² | 0 | 0 | 0 | 0 |
| | chronic back | | | | | | | | | |
| | pain | | | | | | | | | |
| 38 (40/M) | None | T6 | 2 | 1 | 2 | 1.9 × 10³ | 0 | 0 | 0 | 0 |
| 39 (39/M) | None | T6 | 6 | 5 | 5 | 2.7 × 10⁷ | 2 | 3.5 × 10⁵ | 0 | 0 |
| 40 (35/F) | Hypertension | T6-T7 | 9 | 4 | 8 | 7.6 × 10³ | 3 | 6.1 × 10³ | 0 | 0 |
| 41 (59/F) | None | T7 | 3 | 2 | 2 | 2.7 × 10¹ | 0 | 0 | 1 | 0 |
| 42 (23/F) | None | T7-T8 | 6 | 6 | 3 | 2.6 × 10⁴ | 0 | ND | 0 | 0 |
| 43 (50/M) | None | T8 | 5 | 3 | 6 | 1.5 × 10³ | 3 | 0 | 0 | 0 |
| 44 (35/M) | Heart disease | T8 | 14 | 3 | 2 | 6.5 × 10¹ | 0 | 0 | 0 | 0 |
| 45 (72/F) | None | T11 | 5 | 2 | 2 | 2.2 × 10² | 3 | 2.6 × 10³ | 0 | 0 |
| 46 (69/F) | None | T11 | 3 | 2 | 3 | 3.5 × 10¹ | ND | ND | 0 | 0 |
| 47 (65/F) | None | T12 | 2 | 1 | 6 | 5.7 × 10³ | 0 | 0 | 0 | 0 |
| 48 (21/F) | None | T12 | 3 | 0 | 9 | 5.5 × 10³ | ND | ND | ND | ND |
| 49 (51/F) | None | L1 | 2 | 1 | 2 | 2.9 × 10¹ | 0 | 0 | 0 | 0 |
| 50 (58/F) | None | L2-L3 | 4 | 2 | 3 | 4.3 × 10¹ | 0 | 0 | 2 | 0 |
| 51 (72/M) | None | L3 | 1 | 1 | 3 | 1.5 × 10² | ND | ND | 1 | 0 |
| 52 (67/F) | None | L3 | 5 | 1 | 3 | 1.4 × 10² | 0 | 0 | 0 | 0 |
| 53 (44/F) | None | L4-L5 | 21 | 3 | 4 | 4.5 × 10⁴ | 0 | 1.1 × 10² | 0 | 0 |
| 54 (74/M) | None | L5 | 2 | 1 | 2 | 1.4 × 10² | ND | ND | 0 | 0 |

Note: ND, not determined because of failure of patient to return to the clinic for follow-up of pain/salivary VZV DNA determinations.
^{a}Site of zoster: C, cervical; L, lumbar; T, thoracic; V1, ophthalmic division of trigerminal nerve.
^{b}VZV DNA copies/Ml of saliva.

All 54 patients with herpes zoster had rash on day 1, when treatment was started (Table 2). Saliva was obtained from all 54 patients before treatment was started on day 1, from 42 patients on day 8, and from 47 patients on day 15 (not necessarily the same patients). Despite repeated phone calls, some patients did not return for saliva collection but did provide pain scores. Independent of pain score, VZV DNA was detected in saliva from all 54 patients with herpes zoster on day 1 before treatment was started. Linear regression analysis to model log VZV DNA copies on day 1 revealed no significant effect of age (P=0.225) or sex (P=0.652). No HSV DNA was found in saliva from any of the 54 patients with herpes zoster or control subjects, whereas GAPDH DNA was present in all saliva samples.

In 47 (94%) of 50 patients, virus DNA copy numbers decreased in saliva during the 14-day study period, although in 3 patients (6%; patients 6, 31, and 45) virus DNA copy numbers transiently increased before decreasing. Ultimately, virus DNA disappeared from saliva during the 14-day study period in 41 (82%) of the 50 patients. No patients developed any increase in virus DNA copy number after it had begun to decline or disappear from saliva. In 2 patients (patients 16 and 34), virus DNA copy number increased throughout the 14-day study period. In 4 patients (patients 7, 9, 27, and 53), VZV DNA was detected in saliva after pain resolved. There was a significant positive correlation between pain and the presence of VZV DNA in saliva (P<0.0005) as well as between pain and the VZV DNA burden (P<0.0005). Overall, reported pain levels were highest when the VZV copy numbers were high. Furthermore, as VZV DNA disappeared, pain scores decreased.

In the 6 patients with chronic non-dermatomal distribution pain, VZV DNA was not detected in any of 18 saliva samples (3 from each patient) obtained over a 2-week period. During a 6-month follow-up period, none of these patients developed herpes zoster or exhibited an increase in VZV-specific IgG levels (Quest Laboratory). In 14 other healthy control subjects, VZV DNA was not detected in any of 42 saliva samples (3 from each individual) obtained over a 2-week period (data not shown).

Saliva samples from patients 11 and 19 (Table 2) were each inoculated onto subconfluent monolayers of human fetal lung cell fibroblasts and observed for CPE. After one subcultivation, a herpesvirus-specific CPE was observed in cells inoculated with saliva from patient 19 but not in cultures of saliva from patient 11. Both PCR and immunohistochemistry revealed that the CPE was VZV specific (data not shown).

One 21-year-old patient with herpes zoster whose pain preceded rash (patient 48 in table 1) was studied extensively (Table 3). She developed T12-distribution radicular pain (scored as 8) without rash at a time when VZV DNA was detected in both her saliva and plasma; 3 days later, her pain increased to a score of 9, a T12-distribution zoster rash developed, and VZV DNA was again detected in both saliva and plasma. She was treated immediately with oral valacyclovir (1 g 3 times daily for 7 days). Two days after antiviral treatment, the pain level decreased to 7, and VZV was detected in saliva and peripheral blood mononuclear cells (PBMCs). Seven days after antiviral treatment, her pain level was still 7, but VZV DNA was no longer detected in saliva, although it was found in her PBMCs. Three weeks after the onset of pain the patient became pain free, and no VZV DNA was detectable in her saliva.

TABLE 3
Clinical and laboratory profile of a 21-year-old patient
(patient 48 from Table 2) with thoracid-distribution
preherpetic neuralgia followed by zoster

| | | | Days After | | |
|---|---|---|---|---|---|
| | 3 Days | Onset of | Antiviral Treatment | | |
| Parameter | Before Rash^{a} | Rash^{b} | 2 | 7 | 21 |
| Pain Score^{c} | 8 | 9 | 7 | 7 | 0 |
| VZV DNA | | | | | |
| In saliva^{d} | 1.6 × 10¹ | 5.5 × 10³ | 6.5 × 10¹ | 0 | 0 |
| In vesicle fluid | NA | − | + | + | ND |
| In PBMCs | − | − | + | + | ND |
| In plasma | + | + | − | − | ND |

Note:
NA, not applicable;
ND, not determined;
PBMCs, peripheral blood mononuclear cells,
VZV, varicella-zoster virus
^{a}Initial emergency department visit, no rash.
^{b}Sample taken before oral valacyclovir treatment.
^{c}Pain rating on a 10-point scale
^{d}VZV DNA copies/mL of saliva, determined by reatl-time polymerase chain reaction.

Discussion. The diagnosis of herpes zoster was established in 54 patients by the presence of dermatomal distribution rash and pain. When rash developed, every patient was treated immediately with valacyclovir and then studied for 2 weeks. VZV DNA was found in the saliva of all 54 patients. During the 2-week study period, the VZV DNA copy number declined in nearly all patients and disappeared in 82% of the patients. In 2 patients with herpes zoster, 1 of whom (patient 16) was being treated with immunosuppressive drugs for cancer, both salivary virus DNA copy number and pain increased throughout the 14-day study period. In 2 other patients (patients 31 and 45), salivary VZV DNA copy number also increased from day 1 to 8, but both virus and pain disappeared by day 15. In addition to the detection of VZV DNA in saliva from all patients with herpes zoster, infectious VZV was isolated from 1 of 2 patients with herpes zoster whose saliva was cultivated in tissue culture. In contrast, PCR revealed no VZV DNA in saliva sampled identically 3 times over a 2-week period from 6 control subjects with chronic pain or in any of 14 healthy adults. The observed decline in salivary VZV DNA copy number in patients with herpes zoster, matched by a reduction in pain in nearly all patients and the ultimate disappearance of pain in 74% of patients by the end of the 2-week study period, most likely reflects a boost in cell-mediated immune responsiveness to VZV that occurs in adults with herpes zoster (Hayward et al., 1991) combined with oral antiviral treatment.

VZV DNA has been detected in the saliva of patients with Ramsay Hunt syndrome (zoster oticus and peripheral facial palsy) (Furuta et al., 2000; Furuta et al., 2001). Because this syndrome results from virus reactivation in the geniculate ganglion, the detection of VZV DNA in the saliva of such patients is readily explained anatomically since visceral efferent parasympathetic fibers of the seventh cranial nerve pass through the geniculate ganglion before innervating the salivary glands. None of the present patients had geniculate zoster and no known anatomic pathways explain the detection of VZV DNA in the saliva of our patients with zoster in trigeminal, cervical, thoracic, and lumbar dermatomes remote from geniculate ganglia. One possibility might rest in VZV viremia. Infectious VZV has been recovered from blood mononuclear cells (MNCs) of immunosuppressed patients with cancer 2-6 days after zoster (Feldman et al., 1977), VZV has been isolated from the blood of an immunocompetent patient with zoster (Sato et al., 1979), and VZV DNA can be found in blood MNCs 1-23 days after zoster (Gilden et al., 1987; Mainka et al., 1998). Another possibility is that VZV reactivated from geniculate ganglia simultaneously with VZV reactivation from ganglia in the dermatome where zoster occurred. VZV is latent in ganglia at all levels along the human neuraxis. The notion of simultaneous VZV reactivation from multiple ganglia is provided by the classic work of Lewis (1958), who described dermatomal distribution radicular pain in areas distinct from pain with rash, as well as by virological verification of VZV vasculopathy in a dermatome distant from the original site of zoster (Gilden et al., 2002).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
U.S. Pat. No. 4,313,734
U.S. Pat. No. 4,366,241
U.S. Pat. No. 4,632,901
U.S. Pat. No. 4,683,195
U.S. Pat. No. 4,683,202
U.S. Pat. No. 4,770,853
U.S. Pat. No. 4,786,589
U.S. Pat. No. 4,800,159
U.S. Pat. No. 4,816,567
U.S. Pat. No. 4,867,973
U.S. Pat. No. 4,883,750
U.S. Pat. No. 4,965,188
U.S. Pat. No. 5,130,238
U.S. Pat. No. 5,169,766
U.S. Pat. No. 5,279,721
U.S. Pat. No. 5,565,332
U.S. Pat. No. 5,656,448
U.S. Pat. No. 5,840,873
U.S. Pat. No. 5,843,640
U.S. Pat. No. 5,843,651
U.S. Pat. No. 5,846,708
U.S. Pat. No. 5,846,717
U.S. Pat. No. 5,846,726
U.S. Pat. No. 5,846,729
U.S. Pat. No. 5,849,487
U.S. Pat. No. 5,853,990
U.S. Pat. No. 5,853,992
U.S. Pat. No. 5,853,993
U.S. Pat. No. 5,856,092
U.S. Pat. No. 5,861,244
U.S. Pat. No. 5,863,732
U.S. Pat. No. 5,863,753
U.S. Pat. No. 5,866,331
U.S. Pat. No. 5,905,024
U.S. Pat. No. 5,910,407
U.S. Pat. No. 5,912,124
U.S. Pat. No. 5,912,145
U.S. Pat. No. 5,919,630
U.S. Pat. No. 5,925,517
U.S. Pat. No. 5,928,862
U.S. Pat. No. 5,928,869
U.S. Pat. No. 5,929,227
U.S. Pat. No. 5,932,413
U.S. Pat. No. 5,935,791
U.S. Pat. No. 5,843,650
U.S. Pat. No. 5,846,709
U.S. Pat. No. 5,846,783
U.S. Pat. No. 5,849,546
U.S. Pat. No. 5,849,497
U.S. Pat. No. 5,849,547
U.S. Pat. No. 5,858,652
U.S. Pat. No. 5,866,366
U.S. Pat. No. 5,916,776
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 5,843,663
U.S. Pat. No. 5,849,481
U.S. Pat. No. 5,849,486
U.S. Pat. No. 5,851,772.
U.S. Pat. No. 5,872,261
U.S. Pat. No. 5,892,628
U.S. Pat. No. 5,900,481
U.S. Pat. No. 5,912,148
U.S. Pat. No. 5,916,779
U.S. Pat. No. 5,919,626
Ausubel et al., Current Protocols in Molecular Biology, John Wiley & Sons, NY, 1989.
Barany, et al., Proc. Natl. Acad. Sci. USA, 88:189-193, 1991
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Cohrs et al., J. Virol., 74:11464-11471, 2000.
Diggle et al., In: Analysis of longitudinal data, Oxford Science Publn., Clarendon Press, 139-140, 1995.
EP 329,822
EP-A 0 125 118
EP-A 0 143 574
EP-A 0 282 192
EP-A 0 299 428
EP 320 308
Feldman et al., J. Pediatr., 597-600, 1977.
Frohman, In: PCR Protocols: A Guide To Methods And Applications, Academic Press, N.Y., 1990.
Furuta et al., Clin. Infect. Dis., 30:529-533, 2000.
Furuta et al., J. Clin. Microbiol., 39:2856-2859, 2001.
GB Appln. 2 202 328
Gefter et al., Somatic Cell Genet., 3:231-236, 1977.
Gilden et al., Virus Res., 7:117-129, 1987.
Gilden et al., N. Engl. J. Med., 347:1500-1503, 2002.
Goding, In: Monoclonal Antibodies: Principles and Practice, 2d ed., Academic Press, Orlando, Fla., pp 60-61, 71-74, 1986.
Harlow and Lane, In: Antibodies: A Laboratory Manual, Cold Spring Harbor Laboratory, 1988.
Hayward et al., J. Infect. Dis., 163:873-875, 1991.
Innis et al., Proc. Natl. Acad. Sci. USA, 85(24):9436-9440, 1988.
Jacox et al., In: Management of Cancer Pain: Clinical Practice Guideline No. 9, Publn. 94-0592, MD, 1994.
Kendall and Gibbons, In: Rank Correlation Methods, 5^{th} Ed., London, Griffin, 1990.
Kohler and Milstein, Eur. J. Immunol., 6:511-519, 1976.
Kohler and Milstein, Nature, 256:495-497, 1975.
Kwoh et al., Proc. Natl. Acad. Sci. USA, 86:1173, 1989.
Landegren, et al., Science 241:1077-1080, 1988.
Lewis, Lancet., 2:418-421, 1958.
Mainka et al., J. Med., Virol., 56:91-98, 1998.
Mehta et al., J. Med. Virol., 72:174-179, 2004.
Newson, J. Statistical Software, 15:1-10, 2006.
Ohara et al., Proc. Natl. Acad. Sci. USA, 86:5673-5677, 1989.
PCT Appln. WO 87/00880
PCT Appln. WO 88/08534
PCT Appln. WO 88/10315
PCT Appln. WO 89/06700
PCT Appln. WO 90/01069
PCT Appln. PCT/US89/01025
Sambrook and Russell, Molecular Cloning: A Laboratory Manual 3^{rd} Ed., Cold Spring Harbor Laboratory Press, 2001.
Sato et al., Int. J. Oral. Surg., 8:149-154, 1979.
Takekoshi et al., J. Biochem., 130, pp. 299-303, 2001.
Walker et al., Nucleic Acids Res. 20(7):1691-1696, 1992.
Wardemann et al. Science, 301(5638):1374-1377, 2003.

1. A method of detecting an active varicella zoster virus (VZV) infection in a human subject comprising:
(a) obtaining a saliva sample; and
(b) detecting VZV virus particles in said sample.

2. The method of claim 1, wherein detecting comprises an immunoassay.

3. The method of claim 2, wherein said immunoassay comprises use of a first antibody to a first VZV antigen, and use of a second antibody to a second VZV antigen.

4. The method of claim 3, wherein said first antibody is linked to a bead and said second antibody is linked to a detectable marker.

5. The method of claim 4, wherein said bead is sequestered in a reaction chamber, and said detectable marker is detected in said reaction chamber when VZV is present.

6. The method of claim 4, wherein said bead is a magnetic bead.

7. The method of claim 4, wherein said detectable marker is an enzyme, a fluorescent label, or a chemilluminescent label.

8. The method of claim 5, wherein said reaction chamber is a syringe.

9. The method of claim 8, wherein obtaining comprises positioning said syringe in a subject's mouth and filling said syringe with said saliva sample.

10. The method of claim 9, further comprising transferring said saliva sample into a detection chamber.

11. The method of claim 10, wherein said detection chamber is a column.

12. The method of claim 11, wherein said detection chamber is attached to a disposal reservoir.

13. The method of claim 10, further comprising introducing a wash solution into said detection chamber.

14. The method of claim 1, further comprising generating a detectable positive control reaction in said reaction chamber.

15. The method of claim 1, wherein said subject suffers from a recurrent VZV infection.

16. A kit for the detection of an antigen in a fluid comprising:
(a) a reagent chamber comprising a first antibody conjugated to a microbead, and a second antibody conjugated to a detectable marker;
(b) a detection chamber comprising a capture agent for binding said microbead; and
(c) a disposal reservoir;
wherein said reagent chamber and said detection chamber support fluid transfer therebetween, and said detection chamber and said disposal reservoir support fluid transfer therebetween.

17. The kit of claim 16, wherein said first reagent chamber is contained within a syringe.

18. The kit of claim 16, wherein said detection chamber comprises a column.

19. The kit of claim 16, wherein said bead is a magnetic bead, and said capture agent is a metal support.

20. The kit of claim 16, further comprising a wash solution.

21. The kit of claim 20, wherein said wash solution is located in a syringe.

22. The kit of claim 16, further comprising a detection solution.

23. The kit of claim 22, wherein said detection solution is located in a syringe.

24. The kit of claim 16, further comprising a mouth piece that supports operable connection with said reaction chamber.

25. The kit of claim 19, further comprising a magnetic separator.

26. The kit of claim 16, wherein said capture agent binds to a virus.

27. The kit of claim 26, wherein said virus is a herpesvirus.

28. The kit of claim 27, wherein said herpesvirus is varicella zoster virus.

29. The kit of claim 16, wherein said capture agent binds to a bacterium, a fungus or a parasite.

30. The kit of claim 16, wherein said capture agent binds to an environmental or industrial waste product or toxin.

31. The kit of claim 16, wherein said capture agent binds to a bioharzard or bioterrorism agent.
